(19) United States
(12) Patent Application Publication
(10) Pub. No.: US 2026/0357302 A1
(43) Pub. Date: Nov. 12, 2026

(54) FERULIC ACID DECARBOXYLASE AND METHOD FOR PRODUCING UNSATURATED HYDROCARBON COMPOUND USING SAME

(71) Applicants: RIKEN, Wako-shi, Saitama (JP); ZEON CORPORATION, Tokyo (JP); The Yokohama Rubber Co., Ltd., Hiratsuka-shi, Kanagawa (JP)

(72) Inventors: Tomokazu SHIRAI, Wako-shi (JP); Yutaro MORI, Wako-shi (JP); Yoshihide YACHI, Tokyo (JP); Yayoi MATSUMOTO, Hiratsuka-shi (JP); Misao HIZA, Hiratsuka-shi (JP)

(73) Assignees: RIKEN, Wako-shi, Saitama (JP); ZEON CORPORATION, Tokyo (JP); The Yokohama Rubber Co., Ltd., Hiratsuka-shi, Kanagawa (JP)

(21) Appl. No.: 19/123,519

(22) PCT Filed: Oct. 24, 2023
(86) PCT No.: PCT/JP2023/038374

§ 371 Date: Apr. 23, 2025

(30) Foreign Application Priority Data
Oct. 25, 2022 (JP) 2022-170602

(51) Int. Cl. C12P 5/02; C12N 9/88; C12N 15/63

(52) U.S. Cl. CPC C12P 5/026; C12N 9/88; C12N 15/63; C12Y 401/01

(57) ABSTRACT

A combination of (a) FDC with tryptophan at position 397, methionine at position 398, tyrosine at position 440, and methionine at position 189, and/or (b) FDC with tryptophan at position 397, methionine at position 398, tyrosine at position 440, and tryptophan at position 189, and (c) FDC with histidine at position 397 and glutamine at position 398, the catalytic activity for unsaturated hydrocarbon compound production is improved compared to the case where the FDC described in (c) above is used alone, enabling the production of unsaturated hydrocarbon compounds such as butadiene with high productivity.

### TECHNICAL FIELD

The present invention relates to ferulic acid decarboxylase and a method for producing unsaturated hydrocarbon compounds using the same. More specifically, the present invention relates to a ferulic acid decarboxylase having catalytic activity to produce unsaturated hydrocarbon compounds, or a combination of such ferulic acid decarboxylases, a DNA encoding the ferulic acid decarboxylase or the combination, a vector into which the DNA is inserted, and a host cell into which the DNA or the vector is introduced. The present invention also relates to a method for producing unsaturated hydrocarbon compounds using the combination or the host cell. Furthermore, the present invention relates to an agent for promoting production of unsaturated hydrocarbon compounds, comprising the decarboxylase, the DNA, or the vector.

### BACKGROUND ART

It can be said that butadiene (1,3-butadiene) is an extremely important organic compound in the chemical industry because it is used as a raw material for various polymer compounds such as various synthetic rubbers, including butadiene rubber, and polymer resins, including ABS resin. Further, these polymer compounds made from butadiene are widely used not only in industrial products such as automobile tires but also in daily necessities such as clothing. As a result, the demand for butadiene is increasing year by year, with annual demand reaching 13 million tons and a market size of $15 billion.

Traditionally, butadiene has been produced mainly by purifying the C4 fraction produced as a by-product in the production of ethylene and propylene from petroleum. However, due to environmental problems such as the depletion of fossil fuels including petroleum and global warming due to greenhouse gas emissions, there is an increasing need to achieve sustainable butadiene production in order to meet the ever-increasing demand for butadiene mentioned above. Thus, as a countermeasure, a method for producing butadiene from a substance derived from a biomass resource, which is a renewable resource, using an enzyme is being actively developed.

Meanwhile, the present inventors have made it clear that the production of 4-vinylguaiyacol (4VG) by the decarboxylation reaction of ferulic acid, which involves ferulic acid decarboxylase (FDC) (see NPL 1 and the following formula), can be applied to the production of unsaturated hydrocarbon compounds such as butadiene (PTL 3).

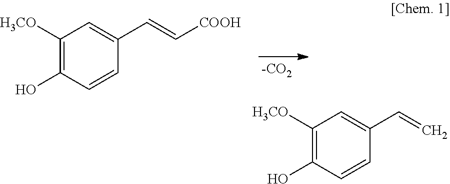

Specifically, it has been found by the present inventors that by introducing a mutation into the amino acid of FDC and changing the substrate specificity of the enzyme from the original ferulic acid to that for muconic acid or the like, butadiene and the like can be produced through a decarboxylation reaction as shown in the following formula (PTLs 1 and 2).

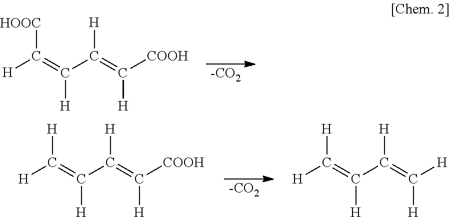

For example, the present inventors have revealed that in a wild-type FDC derived from Saccharomyces cerevisiae (amino acid sequence set forth in SEQ ID NO: 2), FDC mutants in which isoleucine at position 398 is substituted with glutamine or methionine have a catalytic activity improved by 9.3 times and 16.4 times, respectively, compared to the wild-type. Furthermore, the present inventors have also revealed that in the FDC mutant in which the isoleucine at position 398 is substituted with glutamine, when phenylalanine at position 397 is substituted with histidine or methionine, the catalytic activity is improved by 75.1 times and 33.8 times, respectively, compared to the wild-type. The present inventors have also revealed that in the FDC mutant in which the isoleucine at position 398 is substituted with methionine, when phenylalanine at position 397 is substituted with histidine, the catalytic activity is improved by 37.3 times compared to the wild-type (PTL 2).

### CITATION LIST

### Patent Literature

[PTL 1] International Publication No. WO2019/022083
[PTL 2] International Publication No. WO2021/054441

### SUMMARY OF INVENTION

### Technical Problem

An object of the present invention is to provide an enzyme that enables the production of unsaturated hydrocarbon compounds such as butadiene with high productivity.

### Solution to Problem

As described above, the present inventors have revealed that in the wild-type FDC derived from Saccharomyces cerevisiae (amino acid sequence set forth in SEQ ID NO: 2), by substituting isoleucine at position 398 with glutamine and substituting phenylalanine at position 397 with histidine (F397H/1398Q mutant), the catalytic activity is improved by 75.1 times compared to the wild-type (PTL 2).

Therefore, in order to achieve the above object, the present inventors have made intensive studies to further improve the productivity of unsaturated hydrocarbon compounds such as butadiene using such FDC mutants. Specifically, various single or double mutants of FDC were first prepared, and the catalytic activity was evaluated when these mutants were used in combination with the F397H/1398Q mutant.

As a result, it was found that when used in combination with the FDC double mutant (F397Y/1398M or F mutant) in which phenylalanine at position 397 was substituted with tyrosine and isoleucine at position 398 was substituted with methionine or phenylalanine, the catalytic activity was improved by 5.0 times compared to the case where only the F397H/1398Q mutant was used. It was also found that when used in combination with the FDC double mutant (F397W/1398M or F mutant) in which phenylalanine at position 397 was substituted with tryptophan and isoleucine at position 398 was substituted with methionine or phenylalanine, the catalytic activity was improved by 3.4 times compared to the case where only the F397H/1398Q mutant was used.

Furthermore, various FDC triple mutants were prepared by introducing further amino acid substitutions based on the F397Y/1398M or F mutant and the F397W/1398M or F mutant. Then, the combination use with the F397H/1398Q mutant was evaluated.

As a result, it was also found that when phenylalanine at position 440 was substituted with tyrosine in the F397Y/1398M mutant, or when methionine at position 286 was substituted with leucine in the F397Y/1398F mutant, the catalytic activity was further improved beyond the 5-fold increase (5.9 times or 6.2 times).

In addition, when phenylalanine at position 440 was substituted with tyrosine in the F397W/1398M mutant (F397W/I398M/F440Y mutant), or when methionine at position 286 was substituted with leucine in the F397W/1398F mutant (F397W/1398F/M286L mutant), it was also found that the catalytic activity was further improved beyond the 3.4-fold increase (4.6 times or 4.5 times).

Furthermore, various FDC quadruple mutants were prepared by introducing further amino acid substitutions based on these FDC triple mutants. Then, the combination use with the F397H/1398Q mutant was evaluated.

As a result, even when further amino acid substitutions were introduced into the F397Y FDC triple mutant, the improvement did not exceed the 5.9-fold or 6.2-fold increase.

On the other hand, regarding the F397W FDC triple mutant, when isoleucine at position 189 was substituted with methionine in the F397W/1398F/M286L mutant, or when methionine at position 286 was substituted with leucine in the F397W/1398M/F440Y mutant, the catalytic activity was further improved beyond the 4.6-fold or 4.5-fold increase (5.6 times or 5.9 times).

Furthermore, as a result of substituting isoleucine at position 189 with tryptophan or methionine in the F397W/1398M/F440Y mutant, it was revealed that the catalytic activity was improved by about 7 times or more compared to the case where only the F397H/1398Q mutant was used (7.3 times or 8.3 times). That is, it was revealed that since the catalytic activity of the F397H/1398Q mutant alone was 75.1 times higher than that of the wild-type, the catalytic activity when used in combination with the F397W/1398M/F440Y/1189W mutant was improved to about 548 times higher than that of the wild-type, and the catalytic activity when used in combination with the F397W/1398M/F440Y/1189M mutant was improved to about 623 times higher than that of the wild-type.

Thus, the present inventors have succeeded in creating further FDC mutants that can improve the catalytic activity to produce unsaturated hydrocarbon compounds such as butadiene by about 500 times or more compared to the wild-type when used in combination with the F397H/1398Q mutant, and have completed the present invention.

That is, the present invention provides the following aspects.
[1] A combination of at least one ferulic acid decarboxylase selected from the following (a) and (b) and a ferulic acid decarboxylase described in the following (c):
(a) a ferulic acid decarboxylase having, in an amino acid sequence set forth in SEQ ID NO: 2, tryptophan at position 397 or a corresponding position, methionine at position 398 or a corresponding position, tyrosine at position 440 or a corresponding position, and methionine at position 189 or a corresponding position, and having catalytic activity to produce an unsaturated hydrocarbon compound represented by the following formula (2) or (3), or a geometrical isomer thereof;
(b) a ferulic acid decarboxylase having, in the amino acid sequence set forth in SEQ ID NO: 2, tryptophan at position 397 or a corresponding position, methionine at position 398 or a corresponding position, tyrosine at position 440 or a corresponding position, and tryptophan at position 189 or a corresponding position, and having catalytic activity to produce an unsaturated hydrocarbon compound represented by the following formula (2) or (3), or a geometrical isomer thereof;
(c) a ferulic acid decarboxylase having, in the amino acid sequence set forth in SEQ ID NO: 2, histidine at position 397 or a corresponding position and glutamine at position 398 or a corresponding position, and having catalytic activity to produce an unsaturated hydrocarbon compound represented by the following formula (2) or (3), or a geometrical isomer thereof:

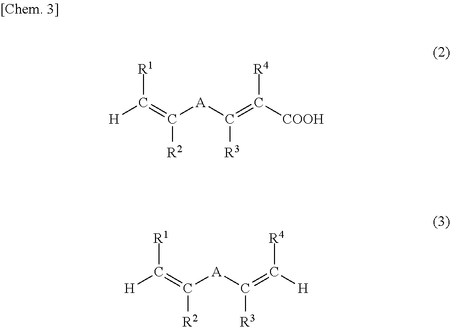

[In formulas (2) and (3), “R¹,” “R²,” “R³,” and “R” each independently represent a hydrogen atom, a linear or branched alkyl group having 1 to 5 carbon atoms, a linear or branched alkoxy group having 1 to 5 carbon atoms, or a hydroxyl group. “A” represents an optionally substituted linear hydrocarbon group having 0 to 5 carbon atoms, and when the number of carbon atoms is 2 to 5, a double bond may be formed between adjacent carbon atoms].
[2] A DNA encoding the combination of ferulic acid decarboxylases according to [1].
[3] A vector comprising a DNA encoding the combination of ferulic acid decarboxylases according to [1].
[4] A combination of a host cell into which a DNA encoding at least one ferulic acid decarboxylase selected from (a) and (b) according to [1], or a vector containing the DNA, is introduced, and a host cell into which a DNA encoding the ferulic acid decarboxylase described in (c) of [1], or a vector containing the DNA, is introduced.
[5] A host cell into which a DNA encoding the combination of ferulic acid decarboxylases according to [1], or a vector containing the DNA, is introduced.
[6] A method for producing an unsaturated hydrocarbon compound represented by the following formula (3) or a geometrical isomer thereof, comprising a step of decarboxylating an unsaturated hydrocarbon dicarboxylic acid compound represented by the following formula (1) or a geometrical isomer thereof in the presence of the combination of ferulic acid decarboxylases according to [1]:

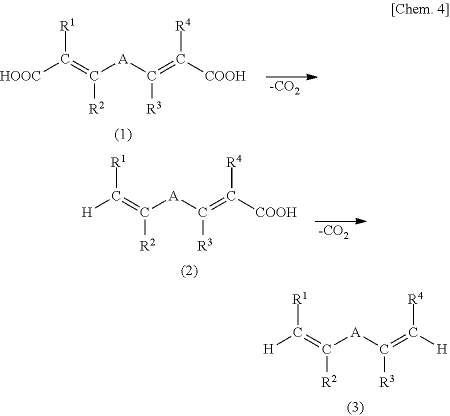

[In formulas (1) to (3), “R¹,” “R²,” “R³,” and “R” each independently represent a hydrogen atom, a linear or branched alkyl group having 1 to 5 carbon atoms, a linear or branched alkoxy group having 1 to 5 carbon atoms, or a hydroxyl group. “A” represents an optionally substituted linear hydrocarbon group having 0 to 5 carbon atoms, and when the number of carbon atoms is 2 to 5, a double bond may be formed between adjacent carbon atoms].
[7] A method for producing an unsaturated hydrocarbon compound, comprising a step of culturing the host cell according to [4] to collect an unsaturated hydrocarbon compound represented by the following formula (3), or a geometrical isomer thereof, produced in the host cell and/or a culture thereof:

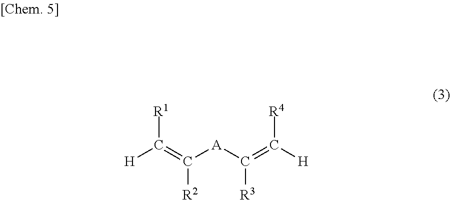

[In formula (3), “R¹,” “R²,” “R³,” and “R” each independently represent a hydrogen atom, a linear or branched alkyl group having 1 to 5 carbon atoms, a linear or branched alkoxy group having 1 to 5 carbon atoms, or a hydroxyl group. “A” represents an optionally substituted linear hydrocarbon group having 0 to 5 carbon atoms, and when the number of carbon atoms is 2 to 5, a double bond may be formed between adjacent carbon atoms].
[8] A method for producing an unsaturated hydrocarbon compound, comprising culturing the combination of host cells according to [5] and collecting the unsaturated hydrocarbon compound represented by formula (3) above, or a geometrical isomer thereof, produced in the combination of host cells and/or a culture thereof.
[9] An agent for promoting production of an unsaturated hydrocarbon compound represented by the following formula (3) or a geometrical isomer thereof by decarboxylating an unsaturated hydrocarbon dicarboxylic acid compound represented by the following formula (1) or a geometrical isomer thereof, comprising the combination of ferulic acid decarboxylases according to [1], a DNA encoding the combination, or a vector containing the DNA:

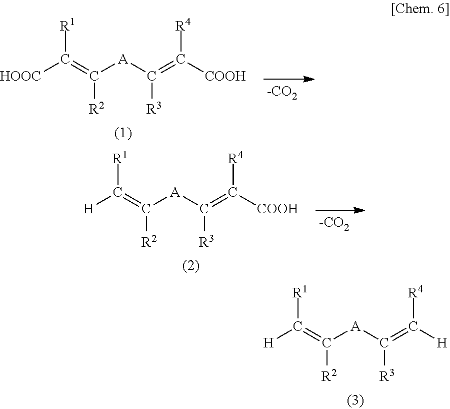

[In formulas (1) to (3), “R¹,” “R²,” “R³,” and “R⁴” each independently represent a hydrogen atom, a linear or branched alkyl group having 1 to 5 carbon atoms, a linear or branched alkoxy group having 1 to 5 carbon atoms, or a hydroxyl group. “A” represents an optionally substituted linear hydrocarbon group having 0 to 5 carbon atoms, and when the number of carbon atoms is 2 to 5, a double bond may be formed between adjacent carbon atoms].
[10] At least one ferulic acid decarboxylase selected from the following (a) and (b):
(a) a ferulic acid decarboxylase having, in an amino acid sequence set forth in SEQ ID NO: 2, tryptophan at position 397 or a corresponding position, methionine at position 398 or a corresponding position, tyrosine at position 440 or a corresponding position, and methionine at position 189 or a corresponding position, and having catalytic activity to produce an unsaturated hydrocarbon compound represented by the following formula (2) or (3), or a geometrical isomer thereof;
(b) a ferulic acid decarboxylase having, in the amino acid sequence set forth in SEQ ID NO: 2, tryptophan at position 397 or a corresponding position, methionine at position 398 or a corresponding position, tyrosine at position 440 or a corresponding position, and tryptophan at position 189 or a corresponding position, and having catalytic activity to produce an unsaturated hydrocarbon compound represented by the following formula (2) or (3), or a geometrical isomer thereof.

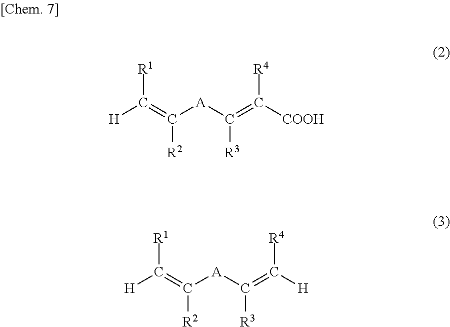

[In formulas (2) and (3), “R¹,” “R²,” “R³,” and “R⁴” each independently represent a hydrogen atom, a linear or branched alkyl group having 1 to 5 carbon atoms, a linear or branched alkoxy group having 1 to 5 carbon atoms, or a hydroxyl group. “A” represents an optionally substituted linear hydrocarbon group having 0 to 5 carbon atoms, and when the number of carbon atoms is 2 to 5, a double bond may be formed between adjacent carbon atoms].
[11] A DNA encoding the ferulic acid decarboxylase according to.
[12] A vector comprising a DNA encoding the ferulic acid decarboxylase according to.
[13] A host cell into which a DNA encoding the ferulic acid decarboxylase according to [10], or a vector containing the DNA, is introduced.
[14] An agent for promoting production of an unsaturated hydrocarbon compound represented by the following formula (3) or a geometrical isomer thereof by decarboxylating an unsaturated d hydrocarbon dicarboxylic acid compound represented by the following formula (1) or a geometrical isomer thereof, comprising the ferulic acid decarboxylase according to [10], the DNA according to, or the vector according to [12], wherein the agent is used in combination with a ferulic acid decarboxylase having, in the amino acid sequence set forth in SEQ ID NO: 2, histidine at position 397 or a corresponding position and glutamine at position 398 or a corresponding position and having the catalytic activity, a DNA encoding the ferulic acid decarboxylase, or a vector containing the DNA:

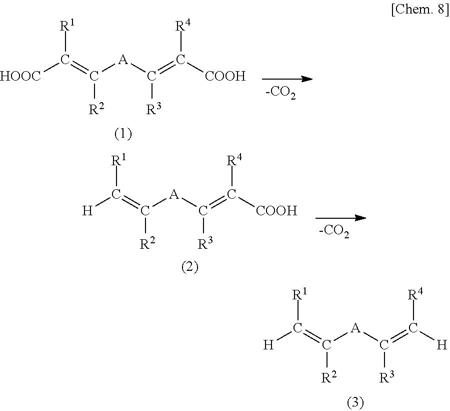

[In formulas (1) to (3), “R¹,” “R²,” “R³,” and “R⁴” each independently represent a hydrogen atom, a linear or branched alkyl group having 1 to 5 carbon atoms, a linear or branched alkoxy group having 1 to 5 carbon atoms, or a hydroxyl group. “A” represents an optionally substituted linear hydrocarbon group having 0 to 5 carbon atoms, and when the number of carbon atoms is 2 to 5, a double bond may be formed between adjacent carbon atoms].

### Advantageous Effects of Invention

The present invention makes it possible to provide an enzyme capable of producing an unsaturated hydrocarbon compound such as butadiene with high productivity, and a method for producing an unsaturated hydrocarbon compound using the enzyme.

### DESCRIPTION OF EMBODIMENTS

### <Ferulic Acid Decarboxylase>

As shown in Examples described later, by using a combination of (a) a ferulic acid decarboxylase having tryptophan at position 397, methionine at position 398, tyrosine at position 440, and methionine at position 189, or (b) a ferulic acid decarboxylase having tryptophan at position 397, methionine at position 398, tyrosine at position 440, and tryptophan at position 189, and (c) a ferulic acid decarboxylase having histidine at position 397 and glutamine at position 398, the catalytic activity promoting the following reaction to produce an unsaturated hydrocarbon compound represented by the following formula (3), or a geometrical isomer thereof, is higher compared to when the ferulic acid decarboxylase described in (c) above is used alone (hereinafter also simply referred to as “catalytic activity to produce unsaturated hydrocarbon compounds”).

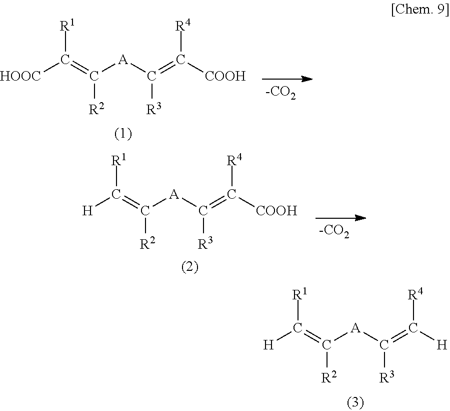

Therefore, the present invention relates to a combination of at least one ferulic acid decarboxylase selected from the following (a) and (b) and a ferulic acid decarboxylase described in the following (c):
(a) a ferulic acid decarboxylase having, in an amino acid sequence set forth in SEQ ID NO: 2, tryptophan at position 397 or a corresponding position, methionine at position 398 or a corresponding position, tyrosine at position 440 or a corresponding position, and methionine at position 189 or a corresponding position, and having the catalytic activity to produce an unsaturated hydrocarbon compound represented by formula (2) or (3) above, or a geometrical isomer thereof;
(b) a ferulic acid decarboxylase having, in the amino acid sequence set forth in SEQ ID NO: 2, tryptophan at position 397 or a corresponding position, methionine at position 398 or a corresponding position, tyrosine at position 440 or a corresponding position, and tryptophan at position 189 or a corresponding position, and having the catalytic activity to produce an unsaturated hydrocarbon compound represented by formula (2) or (3) above, or a geometrical isomer thereof;
(c) a ferulic acid decarboxylase having, in the amino acid sequence set forth in SEQ ID NO: 2, histidine at position 397 or a corresponding position and glutamine at position 398 or a corresponding position, and having the catalytic activity to produce an unsaturated hydrocarbon compound represented by formula (2) or (3) above, or a geometrical isomer thereof.

Note that in the present invention, a “corresponding position” is a position in the same row as isoleucine or the like at position 398 in the amino acid sequence set forth in SEQ ID NO: 2 when the amino acid sequence set forth in SEQ ID NO: 2 is aligned using nucleotide and amino acid sequence analysis software (such as GENETYX-MAC or Sequencher) and BLAST (http://blast.ncbi.nlm.nih.gov/Blast.cgi).

“Ferulic acid decarboxylase (FDC)” is generally an enzyme registered as EC number 4.1.1.102, and refers to an enzyme that catalyzes the following reaction to produce 4-vinylguaiacol (4VG) by decarboxylating ferulic acid.

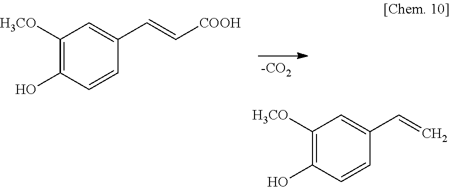

However, in the present invention, as shown in Examples described later, FDC also has catalytic activity to produce an unsaturated hydrocarbon compound represented by formula (2) or (3) above, or a geometrical isomer thereof, by having a specific amino acid at position 397 and 398, etc. The catalytic activity to produce such an unsaturated hydrocarbon compound can be evaluated, for example, by directly measuring the amount of the unsaturated hydrocarbon compound by gas chromatography-mass spectrometry (GC-MS), as shown in Examples described later.

As described above, the FDC of the present invention relates to the embodiment of a combination of at least one FDC selected from (a) and (b) above, and the FDC described in (c) above, and exhibits high catalytic activity to produce unsaturated hydrocarbon compounds compared to the FDC described in (c) above. Here, “high” is preferably 3 times or more, more preferably 4 times or more, further preferably 5 times or more, more preferably 6 times or more, further preferably 7 times or more, and more preferably 8 times or more, compared to the FDC described in (c) above. In addition, the catalytic activity of the FDC of the present invention to produce unsaturated hydrocarbon compounds is preferably 200 times or more, more preferably 300 times or more, further preferably 400 times or more, more preferably 500 times or more, and further preferably 600 times or more, compared to the catalytic activity of the wild-type FDC (for example, a protein consisting of the amino acid sequence set forth in SEQ ID NO: 2 described later) to produce unsaturated hydrocarbon compounds.

The FDC of the present invention, which also has such catalytic activity, includes not only a ferulic acid decarboxylase in which an amino acid at position 397, etc., is artificially substituted with tryptophan or histidine, etc., as shown in Examples described later (hereinafter also simply referred to as “ferulic acid decarboxylase mutant” or “ferulic acid decarboxylase variant”), but also a naturally occurring ferulic acid decarboxylase in which an amino acid at position 397, etc., is tryptophan or histidine, etc. (hereinafter also referred to as “ferulic acid decarboxylase homolog” or “ferulic acid decarboxylase natural mutant”).

The origin of the FDC of the present invention (e.g., Saccharomyces) is not particularly limited, but preferably, a protein that contains an amino acid sequence having an identity of 80% or more (e.g., 81% or more, 82% or more, 83% or more, 84% or more) with the FDC derived from Saccharomyces cerevisiae (amino acid sequence set forth in SEQ ID NO: 2); more preferably, a protein that contains an amino acid sequence having an identity of 85% or more (e.g., 86% or more, 87% or more, 88% or more, 89% or more); further preferably, a protein that contains an amino acid sequence having an identity of 90% or more (e.g., 91% or more, 92% or more, 93% or more, 94% or more); and more preferably, a protein that contains an amino acid sequence having an identity of 95% or more (e.g., 96% or more, 97% or more, 98% or more, 99% or more). In the present invention, “identity” means the percentage (%) of the number of amino acids that match between the FDC and the amino acid sequence set forth in SEQ ID NO: 2, relative to the total number of amino acids of the Saccharomyces-derived FDC.

The FDC of the present invention may also be a protein comprising an amino acid sequence in which one or more amino acids are substituted, deleted, added, and/or inserted at positions other than positions 397 and 398 of the amino acid sequence set forth in SEQ ID NO: 2 (and further at positions 440 and 189 for (a) and (b)). Here, “plural” is not particularly limited, but is usually 2 to 100, preferably 2 to 50, more preferably 2 to 40, further preferably 2 to 30, more preferably 2 to 20, and further preferably 2 to 10 (e.g., 2 to 8, 2 to 4, 2).

The FDC of the present invention may be directly or indirectly added with a different compound. The addition is not particularly limited, and may be an addition at the gene level or a chemical addition. Further, the position to be added is not particularly limited, and may be either the amino terminal (hereinafter also referred to as “N-terminal”) or the carboxyl terminal (hereinafter also referred to as “C-terminal”) of the FDC of the present invention, or both of them. Addition at the gene level is achieved by using a DNA encoding the FDC of the present invention added to a DNA encoding another protein with matching reading frames. The “other proteins” added in this manner are not particularly limited, and for the purpose of facilitating the purification of the FDC of the present invention, tagged proteins for purification such as polyhistidine (His-) tag protein, FLAG-tag protein (registered trademark, Sigma-Aldrich), and glutathione-S-transferase (GST) are preferably used, and further, for the purpose of facilitating the detection of the FDC of the present invention, tagged protein for detection such as fluorescent proteins such as GFP and chemiluminescent proteins such as luciferase are preferably used. The chemical addition may be covalent or non-covalent. The “covalent bond” is not particularly limited, and examples thereof include an amide bond between an amino group and a carboxyl group, an alkylamine bond between an amino group and an alkyl halide group, a disulfide bond between thiols, and a thioether bond between a thiol group and a maleimide group or an alkyl halide group. Examples of the “non-covalent bond” include a biotin-avidin bond. Further, as the “different compound” chemically added in this manner, for the purpose of facilitating the detection of the FDC of the present invention, fluorescent dyes such as Cy3 and rhodamines are preferably used, for example.

Moreover, the FDC of the present invention may be used by mixing with a different component. The different component is not particularly limited, and examples thereof include sterile water, physiological saline, vegetable oils, surfactants, lipids, solubilizer, buffers, protease inhibitors, and preservatives.

### <DNA Encoding FDC and Vector Having the DNA>

The DNA and the like encoding the FDC of the present invention will be described. By introducing such DNA, it becomes possible to transform the traits of a host cell, so that the FDC of the present invention can be produced in the cell, and thus an unsaturated hydrocarbon compound can be produced.

The DNA of the present invention may be a DNA formed by introducing a mutation into a natural DNA, or may be a DNA composed of an artificially designed nucleotide sequence, as long as it encodes the FDC of the present invention described above. Further, the morphology thereof is not particularly limited, and includes cDNA as well as genomic DNA and chemically synthesized DNA. Preparation of these DNAs can be carried out by those skilled in the art using conventional means. A genomic DNA can be prepared by, for example, extracting a genomic DNA from Saccharomyces, preparing a genomic library (examples of vectors available include plasmids, phages, cosmids, BAC, and PAC), developing this, and performing colony hybridization or plaque hybridization using a probe prepared based on the nucleotide sequence of the FDC gene (for example, the nucleotide sequence set forth in SEQ ID NO: 1). It is also possible to prepare a primer specific to the FDC gene and perform PCR using the same. In addition, a cDNA can be prepared by, for example, synthesizing a cDNA based on the mRNA extracted from Saccharomyces, inserting it into a vector such as AZAP to prepare a cDNA library, developing this, and performing colony hybridization or plaque hybridization in the same manner as above, or performing PCR. Then, those skilled in the art can introduce a mutation or the like that substitutes e.g. an amino acid at position 397 of the amino acid sequence set forth in SEQ ID NO: 2 or corresponding to the position with tryptophan or histidine into the DNA thus prepared by using a known position-specific mutation introduction method. Examples of the position-specific mutation introduction method include the Kunkel method (Kunkel, T. A., Proc Natl Acad Sci USA, 1985, Vol. 82, Issue 2, pp. 488 to 492) and the SOE (splicing-by-overlap-extention)-PCR method (Ho, S. N., Hunt, H. D., Horton, R. M., Pullen, J. K., and Pease, L. R., Gene, 1989, Vol. 77, pp. 51 to 59). Further, those skilled in the art can artificially design a nucleotide sequence encoding a protein in which e.g. the amino acid at position 397 of FDC or corresponding to the position is substituted with tryptophan or histidine, and chemically synthesize the DNA of the present invention using an automated nucleic acid synthesizer based on the sequence information.

Furthermore, from the viewpoint of further improving the expression efficiency of the encoded FDC in the host cell, the DNA of the present invention may also take the form of a DNA encoding an FDC of the present invention in which codons are optimized according to the type of the host cell.

Further, the present invention can take the form of the vector inserted with the DNA so that the above-mentioned DNA can be replicated in the host cell. In the present invention, the “vector” can be constructed based on a self-replicating vector, that is, an extrachromosomal independent entity whose replication does not depend on chromosomal replication, for example, a plasmid. The vector may also be one that, when introduced into a host cell, integrates into the genome of the host cell and replicates together with the chromosome into which it has been integrated.

Examples of such a vector include a plasmid and phage DNA. Further, examples of the plasmid include plasmids derived from Escherichia coli (such as PET22, pBR322, pBR325, pUC118, pUC119, pUC18, and pUC19), plasmids derived from yeast (such as YEp13, YEp24, and YCp50), and plasmids derived from Bacillus subtilis (such as pUB110 and pTP5). Examples of the phage DNA include λ phages (such as Charon 4A, Charon 21A, EMBL3, EMBL4, λgt10, λgt11, and AZAP). Further, an insect virus vector such as baculovirus if the host cell is derived from an insect, T-DNA or the like if derived from a plant, and a retrovirus or an adenovirus vector if derived from an animal can also be used as the vector of the present invention. Further, as the procedure and method for constructing the vector of the present invention, those commonly used in the field of genetic engineering can be used. For example, in order to insert the DNA of the present invention into a vector, one employs a method or the like in which a purified DNA is first cleaved with an appropriate restriction enzyme, inserted into the restriction enzyme position or multicloning position of an appropriate vector, and ligated to the vector.

The vector of the present invention may be in the form of an expression vector containing the FDC encoded by the DNA in a state capable of being expressed in the host cell. In order to introduce the “expression vector” into a host cell to express the FDC of the invention, it desirably contains, in addition to the aforementioned DNA, a DNA sequence to control its expression, a genetic marker to select the transformed host cell, and the like. The DNA sequence that controls expression includes a promoter, an enhancer, a splicing signal, a poly A addition signal, a ribosome binding sequence (SD sequence), a terminator, and the like. The promoter is not particularly limited as long as it exhibits transcriptional activity in the host cell, and can be obtained as a DNA sequence that controls the expression of a gene encoding a protein of the same species or heterologous to the host cell. In addition to the DNA sequence that controls the expression, a DNA sequence that induces the expression may be included. Examples of the DNA sequence that induces such expression include, when the host cell is a bacterium, a lactose operon that can induce the expression of a gene located downstream by the addition of isopropyl-β-D-thiogalactopyranoside (IPTG). The gene marker in the present invention may be appropriately selected depending on the method for selecting the transformed host cell, and for example, a gene encoding drug resistance or a gene complementing auxotrophy can be used.

Further, in the present invention, as described above, a plurality of types of FDCs are used in combination; each of these FDCs may be encoded by one vector, or a plurality of FDCs may be encoded by one vector. When one vector encodes a plurality of FDCs, these FDCs can be expressed polycistronically by using, for example, DNA encoding IRES, 2A peptide sequence, or the like.

Moreover, the DNA or vector of the present invention may be used by mixing with different components. The different components are not particularly limited, and examples thereof include sterile water, physiological saline, vegetable oils, surfactants, lipids, solubilizer, buffers, DNase inhibitors, and preservatives.

<Host Cell Introduced with DNA and the Like of the Present Invention>

The host cell into which the DNA or vector of the present invention is introduced will be described. By using a host cell transformed by the introduction of the above-mentioned DNA or vector, it becomes possible to produce the FDC of the present invention, and it becomes also possible to produce an unsaturated hydrocarbon compound represented by the above formula (3), or a geometric isomer thereof.

The host cell into which the DNA or vector of the present invention is introduced is not particularly limited, and examples thereof include microorganisms (such as Escherichia coli, budding yeast, fission yeast, hay bacillus, ray fungi, and filamentous fungi), plant cells, insect cells, and animal cells, and from the viewpoint of showing high proliferation property in a short time with a relatively inexpensive medium, and contributing to the production of unsaturated hydrocarbon compounds represented by the above formula (3), or geometric isomers thereof, which are highly productive, it is preferable to use a microorganism as a host cell, and it is more preferable to use Escherichia coli.

Further, from the viewpoint of inducing prenylation of flavin mononucleotide (FMN) and producing prFMN or an isomer thereof that contributes to the productivity improvement of the unsaturated hydrocarbon compounds represented by the above formula (3), or geometric isomers thereof, the host cell into which the DNA or vector of the present invention is introduced is preferably a cell carrying flavin prenyltransferase.

Further, in the production of butadiene, from the viewpoint that muconic acid, which is a substrate of the FDC of the present invention, can be easily produced from glucose as a raw material, the host cell into which the DNA or vector of the present invention is introduced is preferably a cell in which the pathway for biosynthesizing muconic acid from glucose via 3-dehydroshikimic acid and catechol is activated. Examples of such cells include cells with enzymes that suppress the activity of phosphotransferase-based enzymes and pyruvate kinases and allow the synthesis of aromatic compounds from chorismic acid or isochorismic acid (for example, microorganisms described in International Publication No. 2017/033965) and Escherichia coli, Pseudomonas putida, or budding yeast described in Kruyer N S et al., Curr Opin Biotechnol. 2017, June; 45: pp. 136 to 143.

The introduction of the DNA or vector of the present invention can also be carried out according to the methods commonly used in the art. For example, examples of the method for introducing into a microorganism such as Escherichia coli include a heat shock method, an electroporation method, a spheroplast method, and a lithium acetate method, examples of the method for introducing into plant cells include a method using Agrobacterium and a particle gun method, examples of the method for introducing into insect cells include a method using baculovirus and an electroporation method, and examples of the method for introducing into animal cells include a calcium phosphate method, a lipofection method, and an electroporation method.

The DNA and the like introduced into the host cell in this manner may be retained in the host cell by being randomly inserted into the genomic DNA, may be retained by homologous recombination, and if it is a vector, it can be replicated and retained as an independent entity outside the genomic DNA.

### <Method 1 for Producing Unsaturated Hydrocarbon Compound>

As mentioned above, the FDC of the present invention has a catalytic activity for producing unsaturated hydrocarbon compounds. Therefore, provided is a method for producing an unsaturated hydrocarbon compound represented by the following formula (3) or a geometric isomer thereof, including the steps of decarboxylating an unsaturated hydrocarbon dicarboxylic acid compound represented by the following formula (1) or a geometric isomer thereof in the presence of the FDC of the present invention.

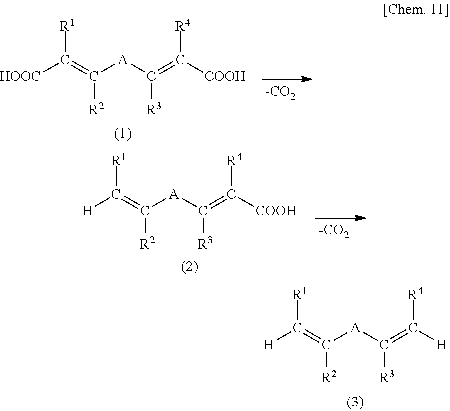

In the present invention, the “unsaturated hydrocarbon compound or a geometric isomer thereof” produced by the reaction means, as shown in the formula (3), a hydrocarbon compound having at least one carbon-carbon double bond, or may have a linear or branched alkyl group having 1 to 5 carbon atoms, a linear or branched alkoxy group having 1 to 5 carbon atoms, or a hydroxyl group introduced therein. Examples of such a compound include butadiene (1,3-butadiene), 2,4-pentadiene acid, isocrotonic acid, 3-methylisocrotonic acid, 3-pentenoic acid, and 10-undecenoic acid.

In the present invention, the “unsaturated hydrocarbon dicarboxylic acid compound or a geometric isomer thereof,” which is a raw material for producing an unsaturated hydrocarbon compound, means, as shown in the above formula (1), a hydrocarbon compound having at least one carbon-carbon double bond and at least two carboxyl groups, or may have a linear or branched alkyl group having 1 to 5 carbon atoms, a linear or branched alkoxy group having 1 to 5 carbon atoms, or a hydroxyl group introduced therein. Examples of such a compound include cis, cis-muconic acid, cis, trans-muconic acid, trans, trans-muconic acid, glutaconic acid, 2-methylglutaconic acid, 3-methylglutaconic acid, and traumatic acid.

Such compounds represented by the formula (1) and geometric isomers thereof can be purchased as commercially available products as shown in Examples described later. Further, those skilled in the art can also synthesize by appropriately referring to a known synthesis method (for example, the method described in Kiyoshi Kudo et al., Journal of the Japan Petroleum Institute, published on Jul. 13, 1994, Vol. 38, Issue 1, pp. 48 to 51).

The “R¹,” “R²,” “R³,” and “R⁴” in the compounds represented by the formulas (1) to (3) and geometric isomers thereof each independently represent a hydrogen atom, a linear or branched alkyl group having 1 to 5 carbon atoms, a linear or branched alkoxy group having 1 to 5 carbon atoms, or a hydroxyl group.

Examples of the “linear or branched alkyl group having 1 to 5 carbon atoms” include a methyl group, an ethyl group, an n-propyl group, an i-propyl group, an n-butyl group, an i-butyl group, an s-butyl group, a t-butyl group, an n-pentyl group, and an i-pentyl group.

Examples of the “linear or branched alkoxy group having 1 to 5 carbon atoms” include a methoxy group, an ethoxy group, an n-propoxy group, an i-propoxy group, an n-butoxy group, an i-butoxy group, an s-butoxy group, a t-butoxy group, an n-pentyloxy group, an i-pentyloxy group, an n-pentyloxy group, and a 1,2-dimethyl-propoxy group.

Further, “A” in the compounds represented by the formulas (1) to (3) and geometric isomers thereof represents an optionally substituted linear hydrocarbon group having 0 to 5 carbon atoms. Note that the an “optionally substituted linear hydrocarbon group having 0 carbon atoms” means that the carbon atoms bonded via “A” are directly bonded to each other without via “A” in the compounds represented by the formulas (1) to (3) and geometric isomers thereof. Further, when the optionally substituted linear hydrocarbon group has 2 to 5 carbon atoms, at least one double bond may be formed between adjacent carbon atoms. Further, examples of the substituent that the hydrocarbon group may have in “A” include a linear or branched alkyl group having 1 to 5 carbon atoms, a linear or branched alkoxy group having 1 to 5 carbon atoms, a hydroxyl group, a halogen atom (such as fluorine, chlorine, bromine, or iodine), a nitro group, a cyano group, an amino group, a carboxyl group, and a formyl group.

The conditions for decarboxylating the unsaturated hydrocarbon dicarboxylic acid compound in the presence of the FDC of the present invention may be any conditions that promotes the decarboxylation and produces the unsaturated hydrocarbon compound, and those skilled in the art can appropriately adjust and set the composition of the reaction solution, the pH of the reaction solution, the reaction temperature, the reaction time, and the like.

For example, the reaction solution added with the FDC of the present invention and a substrate thereof, the unsaturated hydrocarbon dicarboxylic acid compound, is not particularly limited as long as the reaction is not hindered, and is preferably a buffer solution having a pH of 6 to 8, and more preferably a buffer solution containing potassium chloride and sodium phosphate having a pH of 6 to 7. Further, from the viewpoint of more facilitating the reaction, it is preferable to contain a prenylated flavin mononucleotide (prFMN) or an isomer thereof (prFMN^{ketimine} or prFMN^{iminiu}, and for these prFMN and isomers thereof, refer to NPL 1).

The ratio of the FDCs used in the production method of the present invention is not particularly limited, but the molar ratio of the FDC described in (a) above and/or the FDC described in (b) above to the FDC described in (c) above is usually 0.1 to 10 mol/mol, preferably 0.5 to 5 mol/mol, and more preferably 1 to 2 mol/mol.

The reaction temperature is not particularly limited as long as it does not hinder the reaction, but is usually 20 to 40 °C, and preferably 25 to 37 °C. Further, the reaction time may be any time as long as the unsaturated hydrocarbon compound can be produced, and is not particularly limited, but is usually 30 minutes to 7 days, and preferably 12 hours to 2 days.

Further, since the unsaturated hydrocarbon compound produced under such conditions is generally easily vaporized, it can be collected by a known collection and purification method for volatile gas. Examples of such a collecting method include gas stripping, fractional distillation, adsorption, desorption, pervaporation, desorption of isoprene adsorbed on the solid phase from the solid phase by heat or vacuum, extraction with a solvent, or chromatography (such as gas chromatography). Further, even when the produced olefin compound is a liquid, it can also be collected by appropriately using a known collection and purification method (such as distillation or chromatography). Further, these methods may be carried out alone or may be carried out in combination as appropriate in multiple steps.

### <Method 2 for Producing Unsaturated Hydrocarbon Compound>

As mentioned above, by culturing a host cell transformed to express the FDC of the present invention, an unsaturated hydrocarbon compound can be produced with high productivity. Therefore, the present invention also provides a method for producing an unsaturated hydrocarbon compound, including the steps of culturing the host cell into which a DNA or vector encoding the FDC of the present invention is introduced; and collecting an unsaturated hydrocarbon compound represented by the above formula (3) or a geometric isomer thereof produced in the host cell and/or a culture product thereof.

The “host cell into which the DNA or vector encoding the FDC of the present invention is introduced” is as described above, and examples thereof include, as shown in Examples described later, a combination of a host cell into which the DNA encoding at least one FDC selected from (a) and (b) above, or a vector containing the DNA, is introduced, and a host cell into which the DNA encoding the FDC described in (c) above, or a vector containing the DNA, is introduced. In such a combination (co-culture), regarding the ratio of these host cells, when the number of host cells into which the DNA encoding the FDC described in (c) above, or a vector containing the DNA, is introduced is set to 1, the number of host cells into which the DNA encoding at least one FDC selected from (a) and (b) above, or a vector containing the DNA, is introduced is usually 0.1 to 10, preferably from 0.5 to 5, and more preferably from 1 to 2.

Further, the culture of the host cells in the production method of the present invention is not limited to the co-culture described above, but may be a single culture of host cells into which the DNA encoding at least one FDC selected from (a) and (b) above and the DNA encoding the FDC described in (c) above are introduced. In such a case, the ratio of the DNAs (or vectors) introduced into the host cell is not particularly limited, but the molar ratio of the DNA encoding the FDC described in (a) above and/or the DNA encoding the FDC described in (b) above to the DNA encoding the FDC described in (c) above is usually 0.1 to 10 mol/mol, preferably 0.5 to 5 mol/mol, and more preferably 1 to 2 mol/mol.

In addition, the culture conditions for such cells are as described below, and it is preferable that the is added with the unsaturated hydrocarbon dicarboxylic acid compound represented by the above formula (1), which is the substrate of the decarboxylase according to the present invention, or a geometric isomer thereof. The culture temperature may be appropriately changed in design according to the type of host cell used, but is usually 20 to 40 °C, and preferably 25 to 37 °C.

In the present invention, “culture product” refers to a medium containing proliferated host cells, secretory products of the host cells, and metabolic products of the host cells, and the like obtained by culturing host cells in the medium, and includes dilutions and concentrates thereof.

The collection of unsaturated hydrocarbon compounds from such host cells and/or culture products is also not particularly limited, and can be carried out by using the above-mentioned known collection and purification methods. Further, the timing of collection may be any time as long as it is appropriately adjusted according to the type of host cells to be used and an unsaturated hydrocarbon compound can be produced, and is usually 30 minutes to 7 days, and preferably 12 hours to 2 days.

### <Agent for Promoting Production of Unsaturated Hydrocarbon Compound>

As described above, by using the FDC of the present invention, the DNA encoding the FDC, or the vector inserted with the DNA, it is possible to decarboxylate an unsaturated hydrocarbon dicarboxylic acid compound represented by the above formula (1) or a geometric isomer thereof, and promote the formation of unsaturated hydrocarbon compounds represented by the above formula (3) or their geometric isomers.

Therefore, the present invention provides an agent for decarboxylating an unsaturated hydrocarbon dicarboxylic acid compound represented by the above formula (1) or a geometric isomer thereof and promoting production of an unsaturated hydrocarbon compound represented by the above formula (3) or a geometric isomer thereof, including the FDC of the present invention, the DNA encoding the FDC, or the vector inserted with the DNA.

Such an agent may be any one containing the FDC of the present invention or the like, or may be used in mixture with different components. The different components are not particularly limited, and examples thereof include sterile water, physiological saline, vegetable oils, surfactants, lipids, solubilizer, buffers, protease inhibitors, DNase inhibitors, and preservatives.

The present invention can also provide a kit containing such an agent. In the kit of the present invention, the agent may be contained in the form of the host cell transformed due to the introduction of the DNA and the like of the present invention. Further, the kit of the present invention may contain, in addition to such an agent, the compound represented by the above formula (1) or a geometric isomer thereof, a host cell for introducing DNA and the like according to the present invention, a medium for culturing the host cell, an instruction manual for their use, and the like. Further, such a usage instruction manual is an instruction manual for using the agent of the present invention or the like in the above-mentioned method for producing an unsaturated hydrocarbon compound. The instruction manual may include, for example, information about the experimental methods and experimental conditions for the production method of the present invention, the agent of the present invention, and the like (including information such as a vector map showing the nucleotide sequence of a vector and the like, sequence information on an FDC of the present invention, origin and properties of the host cell, and information on culture conditions of the host cell and the like).

### <Method for Producing FDC of the Present Invention>

As shown in Examples described later, by culturing a host cell into which a DNA and the like encoding the FDC of the present invention is introduced, it is possible to produce the FDC in the host cell.

Therefore, the present invention can also provide a method for producing an FDC of the present invention, including the steps of culturing the host cell into which a DNA encoding the FDC of the present invention or a vector including the DNA is introduced; and collecting a protein expressed in the host cell.

In the present invention, the condition of “culturing a host cell” may be any condition as long as the host cell can produce the FDC of the present invention, and those skilled in the art can appropriately adjust and set the temperature, the presence or absence of addition of air, the concentration of oxygen, the concentration of carbon dioxide, the pH of the medium, the culture temperature, the culture time, the humidity, and the like according to the type of the host cell, medium used, and the like.

Such medium may contain anything that can be assimilated by the host cell, and examples of its content include carbon source, nitrogen source, sulfur source, inorganic salts, metal, peptone, yeast extract, meat extract, casein hydrolyzate, serum, and the like. Further, for example, such a medium may be added with IPTG for inducing the expression of the DNA encoding the FDC of the present invention, an antibiotic (such as ampicillin) corresponding to the drug resistance gene that can be encoded by the vector of the present invention, or a nutrient (such as arginine or histidine) corresponding to the gene that complements the auxotrophy that can be encoded by the vector of the invention.

Then, examples of the method for “collecting a protein expressed in the cell” from the host cell cultured in this way include a method in which the host cells are collected from the medium by filtration, centrifugation, or the like, and the collected host cells are treated by cytolysis, grinding treatment, pressure crushing, or the like, and further, the protein expressed in the host cell is purified and concentrated by ultrafiltration treatment, salting out, solvent precipitation such as ammonium sulfate precipitation, chromatography (such as gel chromatography, ion exchange chromatography, or affinity chromatography), or the like. Further, when the above-mentioned purified tagged protein is added to the FDC of the present invention, it can be purified and collected using a substrate to which the tagged protein is adsorbed. Further, these purification and concentration methods may be carried out alone or may be carried out in combination as appropriate in multiple steps.

Further, the FDC of the present invention is not limited to the above biological synthesis, and can also be produced by using the DNA and the like of the present invention and a cell-free protein synthesis system. The cell-free protein synthesis system is not particularly limited, and examples thereof include wheat germ-derived, Escherichia coli-derived, rabbit reticulocyte-derived, and insect cell-derived synthetic systems. Further, those skilled in the art can chemically synthesize the FDC of the present invention using a commercially available peptide synthesizer or the like.

The preferred embodiments of the present invention have been described above, but the present invention is not limited to the above embodiments. For example, as shown in Examples described later, FDC mutants other than the FDCs described in (a) and (b) above, which can improve the catalytic activity to produce unsaturated hydrocarbon compounds by 3 times or more when used in combination with the FDC described in (c) above, have also been revealed. Therefore, in the present invention, not only (a) and (b) above, but also at least one FDC selected from (d) to (m) below can be used.
(d) a ferulic acid decarboxylase having, in the amino acid sequence set forth in SEQ ID NO: 2, tyrosine at position 397 or a corresponding position and methionine at position 398 or a corresponding position, and having the catalytic activity to produce an unsaturated hydrocarbon compound represented by formula (2) or (3) above, or a geometrical isomer thereof;
(e) a ferulic acid decarboxylase having, in the amino acid sequence set forth in SEQ ID NO: 2, tyrosine at position 397 or a corresponding position, methionine at position 398 or a corresponding position, and tyrosine at position 440 or a corresponding position, and having the catalytic activity to produce an unsaturated hydrocarbon compound represented by formula (2) or (3) above, or a geometrical isomer thereof;
(f) a ferulic acid decarboxylase having, in the amino acid sequence set forth in SEQ ID NO: 2, tyrosine at position 397 or a corresponding position and phenylalanine at position 398 or a corresponding position, and having the catalytic activity to produce an unsaturated hydrocarbon compound represented by formula (2) or (3) above, or a geometrical isomer thereof;
(g) a ferulic acid decarboxylase having, in the amino acid sequence set forth in SEQ ID NO: 2, tyrosine at position 397 or a corresponding position, phenylalanine at position 398 or a corresponding position, and leucine at position 286 or a corresponding position, and having the catalytic activity to produce an unsaturated hydrocarbon compound represented by formula (2) or (3) above, or a geometrical isomer thereof;
(h) a ferulic acid decarboxylase having, in the amino acid sequence set forth in SEQ ID NO: 2, tryptophan at position 397 or a corresponding position and methionine at position 398 or a corresponding position, and having the catalytic activity to produce an unsaturated hydrocarbon compound represented by formula (2) or (3) above, or a geometrical isomer thereof;
(i) a ferulic acid decarboxylase having, in the amino acid sequence set forth in SEQ ID NO: 2, tryptophan at position 397 or a corresponding position, methionine at position 398 or a corresponding position, and tyrosine at position 440 or a corresponding position, and having the catalytic activity to produce an unsaturated hydrocarbon compound represented by formula (2) or (3) above, or a geometrical isomer thereof;
(j) a ferulic acid decarboxylase having, in the amino acid sequence set forth in SEQ ID NO: 2, tryptophan at position 397 or a corresponding position, methionine at position 398 or a corresponding position, phenylalanine at position 440 or a corresponding position, and leucine at position 286 or a corresponding position, and having the catalytic activity to produce an unsaturated hydrocarbon compound represented by formula (2) or (3) above, or a geometrical isomer thereof;
(k) a ferulic acid decarboxylase having, in the amino acid sequence set forth in SEQ ID NO: 2, tryptophan at position 397 or a corresponding position and phenylalanine at position 398 or a corresponding position, and having the catalytic activity to produce an unsaturated hydrocarbon compound represented by formula (2) or (3) above, or a geometrical isomer thereof;
(l) a ferulic acid decarboxylase having, in the amino acid sequence set forth in SEQ ID NO: 2, tryptophan at position 397 or a corresponding position, phenylalanine at position 398 or a corresponding position, and leucine at position 286 or a corresponding position, and having the catalytic activity to produce an unsaturated hydrocarbon compound represented by formula (2) or (3) above, or a geometrical isomer thereof;
(m) a ferulic acid decarboxylase having, in the amino acid sequence set forth in SEQ ID NO: 2, tryptophan at position 397 or a corresponding position, phenylalanine at position 398 or a corresponding position, leucine at position 286 or a corresponding position, and methionine at position 189 or a corresponding position, and having the catalytic activity to produce an unsaturated hydrocarbon compound represented by formula (2) or (3) above, or a geometrical isomer thereof.

### EXAMPLES

Hereinafter, the present invention will be described in more detail based on Examples, but the present invention is not limited to the following Examples.

As shown in PTLs 1 and 2, the present inventors have found that introducing mutations with amino acid substitutions into ferulic acid decarboxylase (FDC) improves the catalytic activity for butadiene production using muconic acid as a substrate. In particular, the present inventors have revealed that in the wild-type FDC derived from Saccharomyces cerevisiae (amino acid sequence set forth in SEQ ID NO: 2), by substituting isoleucine at position 398 with glutamine and substituting phenylalanine at position 397 with histidine (F397H/1398Q mutant), the catalytic activity is improved by 75.1 times compared to the wild-type.

Therefore, the present inventors attempted to further improve the productivity of unsaturated hydrocarbon compounds such as butadiene using such FDC mutants; as shown below, various amino acid substitution mutants of FDC were prepared, and the catalytic activity was evaluated when these mutants were used in combination with the F397H/1398Q mutant.

### <Preparation of Plasmid Vector>

First, in order to efficiently express a wild-type FDC derived from Saccharomyces cerevisiae in Escherichia coli, the C-terminal of the wild-type nucleotide sequence encoding them was modified by fusing a polyhistidine tag in consideration of the frequency of codon use in Escherichia coli (the nucleotide sequence after the sequence modification is set forth in SEQ ID NO: 3).

Next, the DNA composed of such a modified nucleotide sequence was chemically synthesized according to a conventional method. Then, the DNA thus prepared and pET22b (+) vectors (manufactured by Novagen) were ligated by the Gibson Assembly method (using a New England Biolabs kit NEBuilder HiFi DNA Assembly Master Mix (registered trademark)) to prepare plasmid vectors (wild-type FDC vectors) capable of expressing the wild-type FDC in Escherichia coli.

Furthermore, using the wild-type FDC vector thus obtained as a template, vectors encoding FDCs with various amino acid substitutions were prepared. Specifically, primers encoding the amino acid sequences into which each mutation was introduced were designed and synthesized. Then, using the wild-type FDC vector as a template and using the primers, according to the protocol of the Gibson Assembly method, plasmid vectors (FDC mutant vectors) capable of expressing in Escherichia coli each FDC into which each mutation was introduced, in a form fused with a polyhistidine tag at its C-terminus, were prepared.

In addition, similarly, the DNA obtained by amplifying a gene (SEQ ID NO: 5) encoding flavin prenyltransferase (hereinafter also referred to as “UbiX”) from an Escherichia coli (K-12) strain by the Polymerase Chain Reaction method and pColADuet vectors (manufactured by Novagen) were ligated by the Gibson Assembly method to prepare plasmid vectors (UbiX vectors) capable of expressing the wild-type UbiX in Escherichia coli.

### <Preparation of Enzyme Reaction Solution and Measurement of Enzyme Activity>

The vectors prepared as described above (5 μg of F397H/1398Q mutant vector or 5 μg of other FDC mutant vector and 5 μg of Ubix vector) were introduced into Escherichia coli C41 (DE3) strain (manufactured by Lucigen Corporation, 100 μL) by the heat shock method to prepare transformants co-expressing F397H/1398Q mutant and UbiX (hereinafter also referred to F397H/1398Q as mutant-expressing Escherichia coli) and transformants co-expressing other FDC mutants and UbiX (hereinafter also referred to as other FDC mutant-expressing Escherichia coli).

Then, each of these transformants was cultured in LB medium supplemented with ampicillin and kanamycin for 6 hours. Note that by this culturing for 6 hours (preculture), the proliferation of these transformants had reached a plateau. Therefore, the quantity of cells at the start of the enzyme reaction described later became uniform among these transformants.

A culture medium for enzyme reaction was prepared by adding lactose to TB medium (12 g/L tryptone, 24 g/L yeast extract, 10 g/L glycerol, 9.4 g/L dipotassium hydrogen phosphate, 2.2 g/L potassium dihydrogen phosphate, 100 mg/L ampicillin, and 50 mg/L kanamycin) to a final concentration of 20 g/L, and further adding cis, cis-muconic acid (manufactured by Sigma-Aldrich), which is a substrate, to a final concentration of 5 g/L.

Then, 100 μL of the culture solution of F397H/1398Q mutant-expressing Escherichia coli cultured for 6 hours, or 50 UL of the culture solution of F397H/1398Q mutant-expressing Escherichia coli and 50 μL of the other culture solution of FDC mutant-expressing Escherichia coli were added to 1 mL of the enzyme reaction medium in a 10 mL vial for headspace gas chromatography-mass spectrometer (HS/GSMS); immediately after the addition, the vial was capped and further cultured at 37 °C with a shaking speed of 180 rpm. The peak area representing the amount of butadiene (1,3-butadiene) produced in the headspace of the vial 18 hours after the start of the culture was measured by GC-MS (product name: GCMS-QP Ultra, manufactured by Shimadzu Corporation). Based on the obtained measurement values, the amount of butadiene produced in the co-culture of F397H/1398Q mutant-expressing Escherichia coli and FDC other mutant-expressing Escherichia coli was compared with that in the single culture of F397H/1398Q mutant-expressing Escherichia coli.

### (Example 1) Combination with FDC Single Mutant or Double Mutant

First, various FDC single mutants or double mutants were prepared, and the amount of butadiene produced when these mutants were used in combination (co-cultured as described above) with the F397H/1398Q mutant was evaluated. The obtained results are shown in Table 1. The numerical values in the table represent the ratio of the butadiene production in each co-culture to that in the single culture of F397H/1398Q mutant-expressing Escherichia coli (the same applies to Tables 2 and 3 below).

TABLE 1

F397

| | | I | L | M | F | Y | W |
|---|---|---|---|---|---|---|---|
| I398 | I | 0.1 | 0.2 | 0.2 | 0.8 | 0.6 | 1.3 |
| | L | 0.1 | 0.1 | 0.1 | 0.6 | 0.4 | 1.1 |
| | M | 0.1 | 2.0 | 1.4 | 3.0 | 5.0 | 3.4 |
| | F | 0.6 | 1.5 | 0.6 | 2.7 | 5.0 | 3.4 |
| | Y | 0.1 | 0.2 | 0.1 | 0.6 | 0.7 | 0.6 |
| | W | 0.1 | 0.1 | 0.1 | 0.2 | 0.2 | 1.4 |

As a result, it was found that when used in combination with the FDC double mutant (F397Y/1398M or F) in which phenylalanine at position 397 was substituted with tyrosine and isoleucine at position 398 was substituted with methionine or phenylalanine, the amount of butadiene produced was improved by 5.0 times compared to the case where only the F397H/1398Q mutant was used. It was also found that when used in combination with the FDC double mutant (F397W/1398M or F) in which phenylalanine at position 397 was substituted with tryptophan and isoleucine at position 398 was substituted with methionine or phenylalanine, the amount of butadiene produced was improved by 3.4 times compared to the case where only the F397H/1398Q mutant was used.

### (Example 2) Combination with FDC Triple Mutant

Furthermore, various FDC triple mutants were prepared by introducing further amino acid substitutions based on the F397Y/1398M or F mutant and the F397W/1398M or F mutant. Then, co-culture was performed as described above, and the combination use with the F397H/1398Q mutant was evaluated. The obtained results are shown in Table 2.

TABLE 2

F397X/I398X′

| | | Y/M | Y/M | W/M | W/F |
|---|---|---|---|---|---|
| L187 | M | 2.5 | 3.0 | 0.8 | 2.1 |
| | F | 1.0 | 1.7 | 1.3 | 1.7 |
| | Y | 1.2 | 2.2 | 1.7 | 1.2 |
| | W | 1.0 | 1.5 | 0.8 | 0.7 |
| I189 | M | 3.8 | 3.8 | 3.3 | 3.0 |
| | F | 1.4 | 1.6 | 1.9 | 2.3 |
| | Y | 1.6 | 1.7 | 2.7 | 2.9 |
| | W | 1.0 | 2.8 | 3.6 | 3.2 |
| M286 | I | 1.4 | 2.1 | 1.2 | 2.0 |
| | L | 2.7 | 6.2 | 1.9 | 4.5 |
| | F | 0.9 | 1.4 | 0.8 | 0.8 |
| | Y | 1.1 | 0.8 | 0.7 | 1.3 |
| | W | 0.6 | 1.5 | 0.9 | 0.6 |
| T326 | M | 0.7 | 0.9 | 0.8 | 0.4 |
| | F | 0.7 | 1.1 | 0.8 | 0.4 |
| | Y | 0.7 | 1.0 | 0.7 | 0.6 |
| | W | 0.7 | 1.1 | 0.7 | 0.4 |
| I330 | M | 1.1 | 1.6 | 0.7 | 0.8 |
| | F | 1.3 | 1.4 | 1.6 | 0.8 |
| | Y | 1.1 | 0.7 | 1.5 | 0.5 |
| | W | 1.0 | 2.0 | 1.6 | 0.6 |
| V334 | M | 2.9 | 1.1 | 2.8 | 0.4 |
| | F | 0.6 | 1.6 | 1.3 | 0.4 |
| | Y | 0.6 | 1.1 | 1.4 | 2.1 |
| | W | 0.6 | 1.3 | 0.8 | 0.4 |
| F440 | M | 1.6 | 3.9 | 1.8 | 3.0 |
| | Y | 5.9 | 2.0 | 4.6 | 2.6 |
| | W | 1.4 | 2.1 | 1.9 | 1.9 |
| L442 | M | 0.8 | 2.2 | 1.3 | 1.0 |
| | F | 1.0 | 3.4 | 1.1 | 0.7 |
| | Y | 1.1 | 2.0 | 1.3 | 0.9 |
| | W | 0.9 | 2.0 | 1.1 | 0.8 |

As a result, it was also found that when phenylalanine at position 440 was substituted with tyrosine in the F397Y/1398M mutant, or when methionine at position 286 was substituted with leucine in the F397Y/1398F mutant, the amount of butadiene produced was further improved beyond the 5-fold increase shown in Example 1 (5.9 times or 6.2 times).

In addition, when phenylalanine at position 440 was substituted with tyrosine in the F397W/1398M mutant (F397W/I398M/F440Y mutant), or when methionine at position 286 was substituted with leucine in the F397W/1398F mutant (F397W/1398F/M286L mutant), it was also found that the butadiene production was further improved beyond the 3.4-fold increase shown in Example 1 (4.6 times or 4.5 times).

### (Example 3) Combination with FDC Quadruple Mutant

Furthermore, various FDC quadruple mutants were prepared by introducing further amino acid substitutions based on these FDC triple mutants. Then, co-culture was performed as described above, and the combination use with the F397H/1398Q mutant was evaluated. The obtained results are shown in Table 3.

TABLE 3

| | | | |
|---|---|---|---|
| Quadruple Mutant | F440M | F440Y | M286L |

| | | | | | |
|---|---|---|---|---|---|
| F397/I398 | Y/M | I189M | 1.6 | 4.7 | 2.5 |
| | | I189W | 0.9 | 3.1 | 1.1 |
| | | M286L | 1.1 | 1.1 | |
| | Y/F | I189M | 2.3 | 1.9 | 4.7 |
| | | I189W | 2.5 | 1.7 | 3.3 |
| | | M286L | 3.8 | 1.5 | |
| | W/M | I189M | 2.9 | 8.3 | 5.6 |
| | | I189W | 3.7 | 7.3 | 4.2 |
| | | M286L | 2.8 | 4.0 | |
| | W/F | I189M | 4.3 | 3.5 | 5.9 |
| | | I189W | 4.4 | 3.1 | 4.0 |
| | | M286L | 2.0 | 2.0 | |

As a result, even when further amino acid substitutions were introduced into the F397Y FDC triple mutant, the improvement did not exceed the 5.9 times or 6.2 times increase shown in Example 2.

On the other hand, regarding the F397W FDC triple mutant, when isoleucine at position 189 was substituted with methionine in the F397W/1398F/M286L mutant, or when methionine at position 286 was substituted with leucine in the F397W/1398M/F440Y mutant, the butadiene production was further improved beyond the 4.6 times or 4.5 times increase shown in Example 2 (5.6 times or 5.9 times).

Furthermore, as a result of substituting isoleucine at position 189 with tryptophan or methionine in the F397W/1398M/F440Y mutant, the amount of butadiene produced improved by about 7 times or more compared to when only the F397H/1398Q mutant was used (7.3 times or 8.3 times). That is, since the butadiene production of the F397H/1398Q mutant alone is 75.1 times higher than that of the wild-type, the amount of butadiene produced when used in combination with the F397W/1398M/F440Y/1189W mutant improved to about 548 times higher than that of the wild-type, and the amount of butadiene produced when used in combination with the F397W/1398M/F440Y/1189M mutant improved to about 623 times higher than that of the wild-type.

Thus, further FDC mutants, which can improve the catalytic activity to produce unsaturated hydrocarbon compounds such as butadiene by about 500 times or more compared to the wild-type by using in combination with the F397H/1398Q mutant, were successfully obtained.

### INDUSTRIAL APPLICABILITY

As described above, according to the present invention, it is possible to provide an enzyme that enables production of unsaturated hydrocarbon compounds such as butadiene with high productivity, and a method for producing unsaturated hydrocarbon compounds using the enzyme. Further, according to the present invention, since unsaturated hydrocarbon compounds can be produced by biosynthesis without chemical synthesis, the environmental burden is small. Therefore, the present invention is extremely useful in the production of raw materials for various synthetic polymers such as synthetic rubber, such as butadiene.

1. A combination of at least one ferulic acid decarboxylase selected from the following (a) and (b) and a ferulic acid decarboxylase described in the following (c):
(a) a ferulic acid decarboxylase having, in an amino acid sequence set forth in SEQ ID NO: 2, tryptophan at position 397 or a corresponding position, methionine at position 398 or a corresponding position, tyrosine at position 440 or a corresponding position, and methionine at position 189 or a corresponding position, and having catalytic activity to produce an unsaturated hydrocarbon compound represented by the following formula (2) or (3), or a geometrical isomer thereof;
(b) a ferulic acid decarboxylase having, in the amino acid sequence set forth in SEQ ID NO: 2, tryptophan at position 397 or a corresponding position, methionine at position 398 or a corresponding position, tyrosine at position 440 or a corresponding position, and tryptophan at position 189 or a corresponding position, and having catalytic activity to produce an unsaturated hydrocarbon compound represented by the following formula (2) or (3), or a geometrical isomer thereof;
(c) a ferulic acid decarboxylase having, in the amino acid sequence set forth in SEQ ID NO: 2, histidine at position 397 or a corresponding position and glutamine at position 398 or a corresponding position, and having catalytic activity to produce an unsaturated hydrocarbon compound represented by the following formula (2) or (3), or a geometrical isomer thereof: 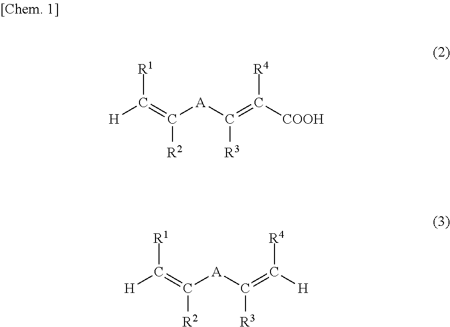
[In formulas (2) and (3), “R¹,” “R²,” “R³,” and “R⁴” each independently represent a hydrogen atom, a linear or branched alkyl group having 1 to 5 carbon atoms, a linear or branched alkoxy group having 1 to 5 carbon atoms, or a hydroxyl group. “A” represents an optionally substituted linear hydrocarbon group having 0 to 5 carbon atoms, and when the number of carbon atoms is 2 to 5, a double bond may be formed between adjacent carbon atoms].

2. A DNA encoding the combination of ferulic acid decarboxylases according to claim 1.

3. A vector comprising a DNA encoding the combination of ferulic acid decarboxylases according to claim 1.

4. A combination of a host cell into which a DNA encoding at least one ferulic acid decarboxylase selected from (a) and (b) according to claim 1, or a vector containing the DNA, is introduced, and a host cell into which a DNA encoding the ferulic acid decarboxylase described in (c) of claim 1, or a vector containing the DNA, is introduced.

5. A host cell into which a DNA encoding the combination of ferulic acid decarboxylases according to claim 1, or a vector containing the DNA, is introduced.

6. A method for producing an unsaturated hydrocarbon compound represented by the following formula (3) or a geometrical isomer thereof, comprising a step of decarboxylating an unsaturated hydrocarbon dicarboxylic acid compound represented by the following formula (1) or a geometrical isomer thereof in the presence of the combination of ferulic acid decarboxylases according to claim 1: 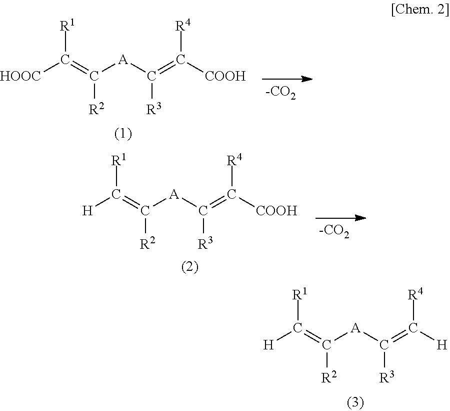
[In formulas (1) to (3), “R¹,” “R²,” “R³,” and “R⁴” each independently represent a hydrogen atom, a linear or branched alkyl group having 1 to 5 carbon atoms, a linear or branched alkoxy group having 1 to 5 carbon atoms, or a hydroxyl group. “A” represents an optionally substituted linear hydrocarbon group having 0 to 5 carbon atoms, and when the number of carbon atoms is 2 to 5, a double bond may be formed between adjacent carbon atoms].

7. A method for producing an unsaturated hydrocarbon compound, comprising a step of culturing a host cell or a combination of host cells to collect an unsaturated hydrocarbon compound represented by the following formula (3), or a geometrical isomer thereof, produced in the host cell and/or a culture thereof, and wherein
the host cell is a host cell into which a DNA encoding the combination of ferulic acid decarboxylases according to claim 1, or a vector containing the DNA, is introduced; and
the combination of host cells is a combination of a host cell into which a DNA encoding at least one ferulic acid decarboxylase selected from (a) and (b) according to claim 1, or a vector containing the DNA, is introduced, and a host cell into which a DNA encoding the ferulic acid decarboxylase described in (c) of claim 1, or a vector containing the DNA, is introduced: 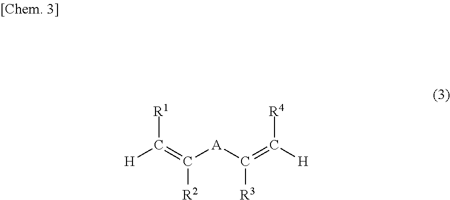
[In formula (3), “R¹,” “R²,” “R³,” and “R⁴” each independently represent a hydrogen atom, a linear or branched alkyl group having 1 to 5 carbon atoms, a linear or branched alkoxy group having 1 to 5 carbon atoms, or a hydroxyl group. “A” represents an optionally substituted linear hydrocarbon group having 0 to 5 carbon atoms, and when the number of carbon atoms is 2 to 5, a double bond may be formed between adjacent carbon atoms].

8. An agent for promoting production of an unsaturated hydrocarbon compound represented by the following formula (3) or a geometrical isomer thereof by decarboxylating an unsaturated hydrocarbon dicarboxylic acid compound represented by the following formula (1) or a geometrical isomer thereof, comprising the combination of ferulic acid decarboxylases according to claim 1, a DNA encoding the combination, or a vector containing the DNA: 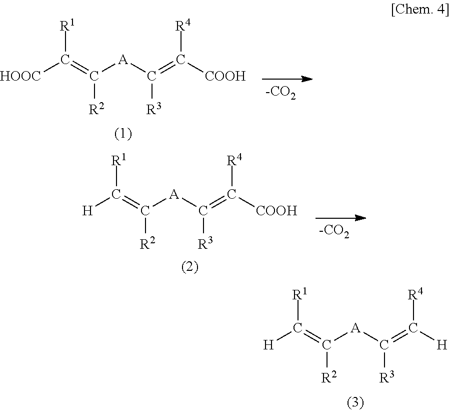
[In formulas (1) to (3), “R¹,” “R²,” “R³,” and “R⁴” each independently represent a hydrogen atom, a linear or branched alkyl group having 1 to 5 carbon atoms, a linear or branched alkoxy group having 1 to 5 carbon atoms, or a hydroxyl group. “A” represents an optionally substituted linear hydrocarbon group having 0 to 5 carbon atoms, and when the number of carbon atoms is 2 to 5, a double bond may be formed between adjacent carbon atoms].

9. At least one ferulic acid decarboxylase selected from the following (a) and (b):
(a) a ferulic acid decarboxylase having, in an amino acid sequence set forth in SEQ ID NO: 2, tryptophan at position 397 or a corresponding position, methionine at position 398 or a corresponding position, tyrosine at position 440 or a corresponding position, and methionine at position 189 or a corresponding position, and having catalytic activity to produce an unsaturated hydrocarbon compound represented by the following formula (2) or (3), or a geometrical isomer thereof;
(b) a ferulic acid decarboxylase having, in the amino acid sequence set forth in SEQ ID NO: 2, tryptophan at position 397 or a corresponding position, methionine at position 398 or a corresponding position, tyrosine at position 440 or a corresponding position, and tryptophan at position 189 or a corresponding position, and having catalytic activity to produce an unsaturated hydrocarbon compound represented by the following formula (2) or (3), or a geometrical isomer thereof. 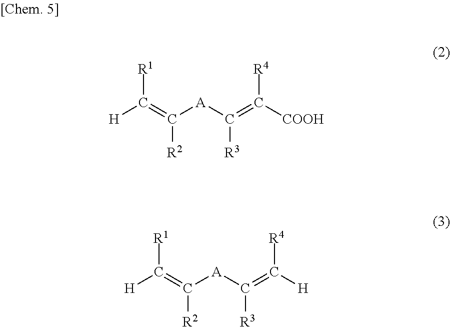
[In formulas (2) and (3), “R¹,” “R²,” “R³,” and “R⁴” each independently represent a hydrogen atom, a linear or branched alkyl group having 1 to 5 carbon atoms, a linear or branched alkoxy group having 1 to 5 carbon atoms, or a hydroxyl group. “A” represents an optionally substituted linear hydrocarbon group having 0 to 5 carbon atoms, and when the number of carbon atoms is 2 to 5, a double bond may be formed between adjacent carbon atoms].

10. A DNA encoding the ferulic acid decarboxylase according to claim 9.

11. A vector comprising a DNA encoding the ferulic acid decarboxylase according to claim 9.

12. A host cell into which a DNA encoding the ferulic acid decarboxylase according to claim 9, or a vector containing the DNA, is introduced.

13. An agent for promoting production of an unsaturated hydrocarbon compound represented by the following formula (3) or a geometrical isomer thereof by decarboxylating an unsaturated hydrocarbon dicarboxylic acid compound represented by the following formula (1) or a geometrical isomer thereof, comprising the ferulic acid decarboxylase according to claim 9, a DNA encoding the ferulic acid decarboxylase according to claim 9, or a vector comprising a DNA encoding the ferulic acid decarboxylase according to claim 9, wherein the agent is used in combination with a ferulic acid decarboxylase having, in the amino acid sequence set forth in SEQ ID NO: 2, histidine at position 397 or a corresponding position and glutamine at position 398 or a corresponding position and having the catalytic activity, a DNA encoding the ferulic acid decarboxylase, or a vector containing the DNA: 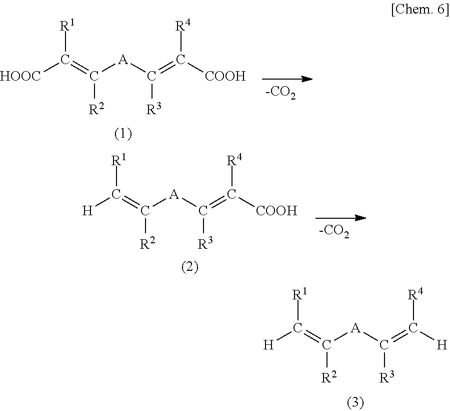
[In formulas (1) to (3), “R¹,” “R²,” “R³,” and “R⁴” each independently represent a hydrogen atom, a linear or branched alkyl group having 1 to 5 carbon atoms, a linear or branched alkoxy group having 1 to 5 carbon atoms, or a hydroxyl group. “A” represents an optionally substituted linear hydrocarbon group having 0 to 5 carbon atoms, and when the number of carbon atoms is 2 to 5, a double bond may be formed between adjacent carbon atoms].


SEQUENCE LISTING (6 sequences)

SEQ ID NO: 1 - DNA, 1512 bp, Saccharomyces cerevisiae
  source 1..1512: mol_type = other DNA
  CDS 1..1512: note = Ferulic acid decarboxylase
atgaggaagctaaatccagctttagaatttagagactttatccaggtcttaaaagatgaagatgacttaatcgaaattaccgaagagattgatccaaatctcgaagtaggtgcaattatgaggaaggcctatgaatcccacttaccagccccgttatttaaaaatctcaaaggtgcttcgaaggatcttttcagcattttaggttgcccagccggtttgagaagtaaggagaaaggagatcatggtagaattgcccatcatctggggctcgacccaaaaacaactatcaaggaaatcatagattatttgctggagtgtaaggagaaggaacctctccccccaatcactgttcctgtgtcatctgcaccttgtaaaacacatatactttctgaagaaaaaatacatctacaaagcctgccaacaccatatctacatgtttcagacggtggcaagtacttacaaacgtacggaatgtggattcttcaaactccagataaaaaatggactaattggtcaattgctagaggtatggttgtagatgacaagcatatcactggtctggtaattaaaccacaacatattagacaaattgctgactcttgggcagcaattggaaaagcaaatgaaattcctttcgcgttatgttttggcgttcccccagcagctattttagttagttccatgccaattcctgaaggtgtttctgaatcggattatgttggcgcaatcttgggtgagtcggttccagtagtaaaatgtgagaccaacgatttaatggttcctgcaacgagtgagatggtatttgagggtactttgtccttaacagatacacatctggaaggcccatttggtgagatgcatggatatgttttcaaaagccaaggtcatccttgtccattgtacactgtcaaggctatgagttacagagacaatgctattctacctgtttcgaaccccggtctttgtacggatgagacacataccttgattggttcactagtggctactgaggccaaggagctggctattgaatctggcttgccaattctggatgcctttatgccttatgaggctcaggctctttggcttatcttaaaggtggatttgaaagggctgcaagcattgaagacaacgcctgaagaattttgtaagaaggtaggtgatatttactttaggacaaaagttggttttatagtccatgaaataattttggtggcagatgatatcgacatatttaacttcaaagaagtcatctgggcctacgttacaagacatacacctgttgcagatcagatggcttttgatgatgtcacttcttttcctttggctccctttgtttcgcagtcatccagaagtaagactatgaaaggtggaaagtgcgttactaattgcatatttagacagcaatatgagcgcagttttgactacataacttgtaattttgaaaagggatatccaaaaggattagttgacaaagtaaatgaaaattggaaaaggtacggatataaataa

SEQ ID NO: 2 - protein, 503 aa, Saccharomyces cerevisiae
MRKLNPALEFRDFIQVLKDEDDLIEITEEIDPNLEVGAIMRKAYESHLPAPLFKNLKGASKDLFSILGCPAGLRSKEKGDHGRIAHHLGLDPKTTIKEIIDYLLECKEKEPLPPITVPVSSAPCKTHILSEEKIHLQSLPTPYLHVSDGGKYLQTYGMWILQTPDKKWTNWSIARGMVVDDKHITGLVIKPQHIRQIADSWAAIGKANEIPFALCFGVPPAAILVSSMPIPEGVSESDYVGAILGESVPVVKCETNDLMVPATSEMVFEGTLSLTDTHLEGPFGEMHGYVFKSQGHPCPLYTVKAMSYRDNAILPVSNPGLCTDETHTLIGSLVATEAKELAIESGLPILDAFMPYEAQALWLILKVDLKGLQALKTTPEEFCKKVGDIYFRTKVGFIVHEIILVADDIDIFNFKEVIWAYVTRHTPVADQMAFDDVTSFPLAPFVSQSSRSKTMKGGKCVTNCIFRQQYERSFDYITCNFEKGYPKGLVDKVNENWKRYGYK

SEQ ID NO: 3 - DNA, 1512 bp, synthetic construct
  misc_feature 1..1512: note = Sequence codon-optimized for E.coli expression
  source 1..1512: mol_type = other DNA
  CDS 1..1512
atgcgtaaactgaatccggcactggaatttcgtgattttattcaggttctgaaagatgaggatgacctgattgaaatcaccgaagaaattgatccgaatctggaagttggtgccattatgcgtaaagcatatgaaagccatctgcctgcaccgctgtttaaaaacctgaaaggtgcaagcaaagacctgtttagcattctgggttgtccggcaggtctgcgtagcaaagaaaaaggtgatcatggtcgtattgcccatcatctgggtttagatccgaaaaccaccatcaaagagatcatcgattatctgctggaatgcaaagagaaagaaccgctgcctccgattaccgttccggttagcagcgcaccgtgtaaaacccatattctgagcgaagaaaaaatccatctgcagagcctgccgacaccgtatctgcatgtttcagatggtggtaaatatctgcagacctatggtatgtggattctgcagacaccggacaaaaaatggaccaattggagcattgcacgtggtatggttgttgatgataaacatattaccggtctggtgattaaaccgcagcatattcgtcagattgcagatagctgggcagcaattggtaaagccaatgaaattccgtttgcactgtgttttggtgttccgcctgcagcaattctggtgagcagcatgccgattccggaaggtgttagcgaaagcgattatgttggtgcgattctgggtgaaagcgttccggtggttaaatgtgaaaccaatgatctgatggttccggcaaccagcgaaatggtttttgaaggcaccctgagcctgaccgatacacatctggaaggtccgtttggtgaaatgcatggttatgtgtttaaaagccagggtcatccgtgtccgctgtataccgttaaagcaatgagctatcgtgataatgcaattctgccggtgagcaatccgggtctgtgtaccgatgaaacccatacactgattggtagcctggttgccaccgaagcaaaagaactggcaattgaaagcggtctgccgattctggatgcatttatgccgtatgaagcacaggcactgtggctgattctgaaagttgatctgaaaggactgcaggcactgaaaaccacaccggaagaattttgtaaaaaagtgggcgatatctacttccgtaccaaagttggttttatcgtgcatgaaattattctggtggccgatgatatcgacatcttcaactttaaagaagtgatttgggcatacgtgacccgtcatacaccggttgcagatcagatggcatttgatgatgttaccagctttccgctggcaccgtttgttagccagagcagccgtagcaaaacaatgaaaggtggcaaatgtgtgaccaactgtatttttcgtcagcagtatgaacgcagcttcgattatatcacctgtaactttgaaaagggctatccgaaaggtctggtggataaagttaatgaaaactggaaacgctacggctataaataa

SEQ ID NO: 4 - protein, 503 aa, synthetic construct; identical to SEQ ID NO: 2
  REGION 1..503: note = Synthetic Construct

SEQ ID NO: 5 - DNA, 570 bp, Escherichia coli
  source 1..570: mol_type = other DNA
  CDS 1..570: note = Flavin prenyltransferase
atgaaacgactcattgtaggcatcagcggtgccagcggcgcgatttatggcgtgcgcttattacaggttctgcgcgatgtcacagatatcgaaacgcatctggtgatgagccaggcagcgcgccagaccttatccctcgaaacggatttttctctgcgcgaagtgcaggcattagccgatgtcacgcacgatgcgcgcgatattgccgccagcatctcttccggttctttccagacgctggggatggtgattttaccctgttcaatcaaaaccctttccggcattgtccatagctatactgatggcttactgacccgtgcggcagatgtggtgctgaaagagcgtcgcccgttggtgctctgcgtgcgtgaaacaccattgcacttaggccatctgcgtttaatgactcaggcggcagaaatcggtgcggtgattatgcctcccgttccggcgttttatcatcgcccgcaatcccttgatgatgtgataaatcagacggttaatcgtgttcttgaccagtttgcgataacccttcctgaagatctctttgcccgctggcagggcgcataa

SEQ ID NO: 6 - protein, 189 aa, Escherichia coli
MKRLIVGISGASGAIYGVRLLQVLRDVTDIETHLVMSQAARQTLSLETDFSLREVQALADVTHDARDIAASISSGSFQTLGMVILPCSIKTLSGIVHSYTDGLLTRAADVVLKERRPLVLCVRETPLHLGHLRLMTQAAEIGAVIMPPVPAFYHRPQSLDDVINQTVNRVLDQFAITLPEDLFARWQGA